(19) United States
(12) Patent Application Publication
(10) Pub. No.: US 2022/0033331 A1
(43) Pub. Date: Feb. 3, 2022

(54) SHUT-DOWN PROCESS FOR THE PRODUCTION OF GLYCOLS

(71) Applicant: SHELL OIL COMPANY, HOUSTON, TX (US)

(72) Inventors: Dionysius Jacobus Maria DE VLIEGER, Amsterdam (NL); Pieter HUIZENGA, Amsterdam (NL); Munro MACKAY, Amsterdam (NL); Lucas Petrus Simon KEYZER, Amsterdam (NL); Duraisamy MUTHUSAMY, Houston, TX (US)

(21) Appl. No.: 17/277,261

(22) PCT Filed: Sep. 10, 2019
(86) PCT No.: PCT/US2019/050309

§ 371 Date: Mar. 17, 2021

Related U.S. Application Data
(60) Provisional application No. 62/730,576, filed on Sep. 13, 2018.

(51) Int. Cl. C07C 29/132; B01J 23/30; B01J 19/00; B01J 8/00

(52) U.S. Cl. CPC C07C 29/132; B01J 23/30; B01J 2219/00245; B01J 8/0015; B01J 2208/00637; B01J 19/0013

(57) ABSTRACT

The invention provides a shut down method for a process for the preparation of glycols from a starting material comprising one or more saccharides in the presence 5 of hydrogen and a catalyst system comprising one or more retro-aldol catalysts comprising tungsten and one or more catalytic species suitable for hydrogenation in a reactor, said method comprising removing the one or more retro-aldol catalysts from the reactor whilst also in the presence of one or more agents suitable to suppress tungsten precipitation.

The present application claims the benefit of U.S. Provisional Application No. 62/730,576, filed Sep. 13, 2018.

### FIELD OF THE INVENTION

The present invention relates to a shut-down procedure for a process for the preparation of ethylene and propylene glycols from saccharide-containing feedstocks.

### BACKGROUND OF THE INVENTION

Monoethylene glycol (MEG) and monopropylene glycol (MPG) are valuable materials with a multitude of commercial applications, e.g. as heat transfer media, antifreeze, and precursors to polymers, such as PET. Ethylene and propylene glycols are typically made on an industrial scale by hydrolysis of the corresponding alkylene oxides, which are the oxidation products of ethylene and propylene, produced from fossil fuels.

In recent years, increased efforts have focused on producing chemicals, including glycols, from renewable feedstocks, such as sugar-based materials. The conversion of sugars to glycols can be seen as an efficient use of the starting materials with the oxygen atoms remaining intact in the desired product.

Current methods for the conversion of saccharides to glycols revolve around a hydrogenation/hydrogenolysis process as described in Angew. Chem. Int. Ed. 2008, 47, 8510-8513.

A preferred methodology for a commercial scale process would be to use continuous flow technology, wherein feed is continuously provided to a reactor and product is continuously removed therefrom. By maintaining the flow of feed and the removal of product at the same levels, the reactor content remains at a more or less constant volume.

Continuous flow processes for the production of glycols from saccharide feedstock have been described in US20110313212, CN102675045, CN102643165, WO2013015955 and CN103731258. A process for the co-production of bio-fuels and glycols is described in WO2012174087.

Typical processes for the conversion of saccharides to glycols require two catalytic species in order to catalyze retro-aldol and hydrogenation reactions. Typically, the hydrogenation catalyst compositions tend to be heterogeneous. However, the retro-aldol catalysts are generally homogeneous in the reaction mixture. Such catalysts are inherently limited due to solubility constraints. Further, the saccharide-containing feedstock is generally in the form of a slurry in a solvent or as a homogeneous saccharide solution.

The homogeneous tungsten-based catalysts typically used in a saccharides to glycols process may be susceptible to conversion to undesirable products, for example by reduction and precipitation of the metal (tungsten). Precipitated solids in a reactor system can lead to blocked lines and clogging as well as undesirable chemical and/or physical reactions of the tungsten metal with other species present (e.g. catalyst poisoning).

The deposition may occur on any surface, including the walls of the reactor and the surface of the solid hydrogenation catalyst. Over time, this deposition can lead to reduced product yields, operational upsets and reduced catalyst performance

It is desirable to provide an improved shut-down procedure to the process for the conversion of saccharides to glycols in which the deposition of the retro-aldol catalysts is minimized or eliminated.

### SUMMARY OF THE INVENTION

In some embodiments, a shut-down method is described for a process for the preparation of glycols from a stream including one or more saccharides in the presence of hydrogen and a catalyst system including one or more retro-aldol catalysts comprising tungsten and one or more catalytic species suitable for hydrogenation in a reactor, said method including removing the one or more retro-aldol catalysts from the reactor whilst also in the presence of one or more agents suitable to suppress tungsten precipitation.

In another embodiment, a shutdown process is described for the preparation of monoethylene glycol from a stream including one or more saccharides in the presence of hydrogen and a catalyst system including one or more tungsten based retro-aldol catalysts in a reactor having one or more catalytic species suitable for hydrogenation, said process including: reducing the reactor temperature to less than 160° C.; removing the one or more tungsten based retro-aldol catalysts from the reactor; and removing the one or more agents suitable to suppress tungsten precipitation from the reactor.

### DETAILED DESCRIPTION OF THE INVENTION

The inventors have surprisingly found that retro-aldol catalyst deposition during the shut-down procedure may be prevented by the presence of at least one agent suitable to suppress catalyst deposition in the reactor when the retro-aldol catalyst is removed from the reactor. The retro-aldol catalysts may be removed prior to the removal of the at least one agent suitable to suppress catalyst deposition or may be removed concurrently with the at least one agent suitable to suppress catalyst deposition.

The shut-down process of the present invention allows increased lifetime of catalyst and longer operation of the process. In particular, the present invention minimizes or eliminates the retro-aldol catalyst deposition in the shut-down process.

The shut-down process of the present invention provides that at least one agent suitable to suppress catalyst deposition is present with the retro-aldol catalyst when removing the retro-aldol catalyst from the reactor.

According to some embodiments of the invention, a method for producing ethylene glycol from a carbohydrate feed may include contacting, in a reactor under hydrogenation conditions, the carbohydrate feed with a bi-functional catalyst system.

Examples of the saccharide feed may include or be derived from at least one saccharide selected from the group consisting of monosaccharides, disaccharides, oligosaccharides and polysaccharides. Saccharides, also referred to as sugars, carbohydrates or organic oxygenates, comprise monomeric, dimeric, oligomeric and polymeric aldoses, ketoses, or combinations of aldoses and ketoses, the monomeric form comprising at least one alcohol and a carbonyl function, being described by the general formula of CₙH₂ₙOₙ (n=4, 5 or 6). Typical C₄ monosaccharides comprise erythrose and threose, typical C5 saccharide monomers include xylose and arabinose and typical C₆ sugars comprise aldoses like glucose, mannose and galactose, while a common C₆ ketose is fructose. Examples of dimeric saccharides, comprising similar or different monomeric saccharides, include sucrose, maltose and cellobiose. Saccharide oligomers are present in corn syrup. Polymeric saccharides include cellulose, starch, glycogen, hemicellulose, chitin, and mixtures thereof.

If the saccharide feed used includes or is derived from oligosaccharides or polysaccharides, it is preferable that it is subjected to pre-treatment before being used in the process of the present invention. Suitable pre-treatment methods are known in the art and one or more may be selected from the group including, but not limited to, sizing, drying, grinding, hot water treatment, steam treatment, hydrolysis, pyrolysis, thermal treatment, chemical treatment, biological treatment. However, after said pre-treatment, the saccharide feed still comprises mainly monomeric and/or oligomeric saccharides. Said saccharides are, preferably, soluble in the reaction solvent.

Preferably, the saccharide feed, after any pre-treatment, comprises saccharides selected from glucose, starch and/or hydrolyzed starch. Hydrolyzed starch comprises glucose, sucrose, maltose and oligomeric forms of glucose. Said saccharides are suitably present as a solution, a suspension or a slurry in a first solvent.

The first solvent may be water or a C₁ to C₆ alcohol or polyalcohol (including sugar alcohols), ethers, and other suitable organic compounds or mixtures thereof. Preferred C₁ to C₆ alcohols include methanol, ethanol, 1-propanol and iso-propanol. Polyalcohols of use include glycols, particularly products of the hydrogenation/retro-aldol reaction, glycerol, erythritol, threitol, sorbitol and mixtures thereof. Preferably, the first solvent comprises water.

The saccharide feed is contacted with a bi-functional catalyst system in a reactor. The bi-functional catalyst system may include a heterogeneous hydrogenation catalyst and a soluble retro-aldol catalyst. In some embodiments, the reactor is filled with the heterogeneous hydrogenation catalytic composition. The weight ratio of the hydrogenation catalyst composition (based on the amount of metal in said composition) to the saccharide feed is suitably in the range of from 10:1 to 1:100. Said hydrogenation catalyst composition is preferably heterogeneous and is retained or supported within the reactor vessel. Further, said hydrogenation catalytic composition also preferably includes one or more materials selected from transition metals from groups 8, 9 or 10 or compounds thereof, with catalytic hydrogenation capabilities.

More preferably, the hydrogenation catalytic composition comprises one or more metals selected from the list consisting of iron, cobalt, nickel, ruthenium, rhodium, palladium, iridium and platinum. This metal or metals may be present in elemental form or as compounds. It is also suitable that this component is present in chemical combination with one or more other ingredients in the hydrogenation catalytic composition. It is required that the hydrogenation catalytic composition has catalytic hydrogenation capabilities and it is capable of catalyzing the hydrogenation of material present in the reactor.

In one embodiment, the hydrogenation catalytic composition comprises metals supported on a solid support. In this embodiment, the solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

Alternatively, the heterogeneous hydrogenation catalytic composition may be present as Raney material, such as Raney nickel, preferably present in a pelletized form.

The soluble retro-aldol catalyst composition preferably comprises one or more compound, complex or elemental material comprising tungsten, molybdenum, vanadium, niobium, chromium, titanium or zirconium. More preferably the retro-aldol catalyst composition comprises one or more material selected from the list consisting of tungstic acid, molybdic acid, ammonium tungstate, ammonium metatungstate, ammonium paratungstate, tungstate compounds comprising at least one Group I or II element, metatungstate compounds comprising at least one Group I or II element, paratungstate compounds comprising at least one Group I or II element, heteropoly compounds of tungsten, heteropoly compounds of molybdenum, tungsten oxides, molybdenum oxides, vanadium oxides, metavanadates, chromium oxides, chromium sulfate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the retro-aldol catalyst composition comprises one or more compound, complex or elemental material selected from those containing tungsten or molybdenum.

In some embodiments, the retro-aldol catalyst is a tungsten-based retro-aldol catalytic species and an alkali metal containing species in a second solvent, making up a retro-aldol stream.

The second solvent is preferably selected from C₁ to C₆ alcohols or polyalcohols (including sugar alcohols), ethers, and other suitable organic compounds or mixtures thereof. Polyalcohols of use include glycols, particularly products of the hydrogenation/retro-aldol reaction, glycerol, erythritol, threitol, sorbitol and mixtures thereof.

The alkali metal in the alkali metal containing species is preferably lithium, sodium or potassium, more preferably sodium. Further, the alkali metal containing species is preferably present as or derived from a buffer, and/or any other component used to control or modify pH, and/or the tungsten-based retro-aldol catalytic species present in the reactor system.

The weight ratio of the metal-based retro-aldol catalytic species (based on the amount of metal in said composition) to sugar in the combined feed stream is suitably in the range of from 1:1 to 1:1000. In some embodiments, the combined feed stream includes the saccharide feed and one or more agents suitable to suppress catalyst deposition.

The molar ratio of alkali metal:metal in the combined feed stream is maintained in the range of from 0.55 to 6.0. Preferably, the molar ratio of alkali metal:metal in the combined feed stream is maintained in the range of from 0.55 to 3.0, more preferably in the range of from 1.0 to 2.0.

The retro-aldol stream is at a temperature in the range of from 150° C. to 250° C. Preferably, the temperature of the retro-aldol stream is no more than 230° C. Preferably, the temperature of the retro-aldol stream is at least 160° C. In one preferred embodiment the retro-aldol stream is maintained at a temperature of no more than 10° C. below the temperature in the reactor system.

In some embodiments, one or more agents suitable to suppress catalyst deposition are also continuously fed to the reactor. The agents suitable to suppress catalyst deposition should be in sufficient quantities to suppress deposition when the retro-aldol catalyst is within the reactor. The concentration of agents suitable to suppress catalyst deposition depends on many parameters, such as, but not limited to, at least one of feedstock concentration, organic oxygenates in the product concentration, retro-aldol catalyst concentration, pH, temperature, pressure, etc. In some embodiments, the concentration of the agents suitable to suppress catalyst deposition may be equal to the organic oxygenates concentration during normal operation of the process which are typically from about 20 to about 40 weight percent. In some embodiments, the organic oxygenates concentration during normal reactor operation will be a combination of the organic oxygenates in the reactor and any organic oxygenates in the agents suitable to suppress catalyst deposition. In some embodiments, lower concentrations of organic oxygenates may be suitable. In other embodiments, a higher concentration of organic oxygenates may be suitable.

Examples of the agents suitable to suppress catalyst deposition may include the saccharide feed or products formed during the process, e.g. sorbitol, MEG, MPG, 1,2-butanediol, glycerol, other sugar alcohols, aldehydes, ketones, carboxylic acids (glycolic acid, lactic acid, acetic acid), etc. In other embodiments, the agents suitable to suppress catalyst deposition may include adipic acid, sodium bicarbonate, sodium hydroxide, sodium adipate, sodium acetate, sodium lactate, and sodium glycolate. These agents may be recycled back to the reactor to maintain the concentration of the agents suitable to suppress catalyst deposition in the reactor.

In other embodiments, the agents suitable to suppress catalyst deposition may include buffer agents which are typically used during the process to control the pH. The buffer agents include organic acids and their corresponding conjugated bases with alkali-metal as their counterions. Examples of suitable buffers include, but are not limited to, acetate buffers, phosphate buffers, lactate buffers, glycolate buffers, citrate buffers and buffers of other organic acids. In a preferred embodiment of the invention, the buffers are alkali metal, more preferably potassium, lithium or sodium, even more preferably sodium species. In other embodiments, the agents suitable to suppress catalyst deposition may include organic acids such as, but not limited to, acetic acid, lactic acid, glycolic acid, glyoxylic acid, oxalic acid, acrylic acid, pyruvic acid, malonic acid, propanoic acid, glyceric acid, maleic acid, butanoic acid, methyl melanoic acid, malic acid, tartaric acid, dihydroxytartaric acid, itaconic acid, mesaconic acid, glutaric acid, dimethylmalonic acid, pentanoic acid, citric acid, adipic acid, and hexanoic acid. In some embodiments, the organic acids are those produced in the process and which can be recycled via the organic oxygenates stream.

Hydrogen is also present in the reactor with the retro-aldol catalytic composition.

The disclosed method for producing glycols from a carbohydrate feed may be performed under particular hydrogenation conditions. For example, the hydrogenation conditions may include temperature, pressure, flow rate, and any other process variable that may be controlled. In an embodiment, the hydrogenation conditions may include a temperature in the range of from 180-250° C. and from 210-250° C. The hydrogenation conditions may also include a pressure in the range of from 500 to 2000 psig.

Once the process for producing glycols is determined to be shut-down, for maintenance, catalyst changeout, etc. care should be taken to lower the chances of tungsten precipitation. In some embodiments, the retro-aldol catalyst should be removed from the reactor prior to the feed and/or the one or more agents suitable to suppress catalyst deposition.

As the retro-aldol catalyst is removed from the reactor, the temperature and the pressure of the reactor are decreased. The temperature during shut-down in the reactor is suitably at least 120° C., preferably at least 130° C., more preferably at least 140° C., most preferably at least 150° C. The lower temperature ensures the thermal degradation of the agents suitable to suppress catalyst deposition or the saccharide feed is negligible in the presence of the hydrogenation catalyst during the shut-down process. The pressure in the reactor during shut-down is suitably at least 1 MPa, preferably at least 2 MPa, more preferably at least 3 MPa. The pressure in the reactor during startup is suitably at most 12 MPa, preferably at most 10 MPa, more preferably at most 8 MPa. In some embodiments, the pressure in the reactor during shut-down may be in the range from 1 MPa to 12 MPa, from 2 MPa to 10 MPa or from 3 MPa to 8 MPa.

Once the temperature and pressure have been decreased, the retro-aldol catalyst may be removed from the reactor while the saccharide feed and/or the one or more agents suitable to suppress catalyst deposition in the reactor are continued to be fed to the reactor. In other embodiments, the saccharide feed and/or the one or more agents suitable to suppress catalyst deposition may be removed along with the retro-aldol catalyst. In still other embodiments, the one or more agents suitable to suppress catalyst deposition may be removed along with the retro-aldol catalyst while continuing to feed the saccharide feed to the reactor. If the agents suitable to suppress catalyst deposition are removed concurrently with the retro-aldol catalyst, there should be a sufficient concentration of the saccharide feed suitable to suppress catalyst deposition in the reactor. If the retro-aldol catalyst is removed prior to the saccharide feed and the one or more agents suitable to suppress catalyst deposition, once the retro-aldol catalyst has been removed from the reactor, the saccharide feed and the one or more agents suitable to suppress catalyst deposition may be stopped and the reactor taken out of operation.

In some embodiments, the agents suitable to suppress catalyst deposition may be removed from the reactor prior to the removal of the retro-aldol catalyst. However, the hydrocarbon content in the reactor via the saccharide feed should be maintained at a sufficient concentration suitable to suppress catalyst deposition in the reactor.

It is postulated, without wishing to be bound by theory, that the presence of agents suitable to suppress catalyst deposition in the reactor along with the retro-aldol catalyst prevents the deposition of the retro-aldol catalyst. Precipitation in the reactor could result in operational issues (e.g. clogging) or uncontrollable chemistry in the reactor (Side reactions catalyzed by the precipitated tungsten).

One of the implications is that during shut-down of the glycol process, the presence of agents suitable to suppress catalyst deposition will suppress the deposition of the retro-aldol catalyst while the retro-aldol catalyst is being removed from the reactor.

1. A shut-down method for a process for the preparation of glycols from a stream comprising one or more saccharides in the presence of hydrogen and a catalyst system comprising one or more retro-aldol catalysts comprising tungsten and one or more catalytic species suitable for hydrogenation in a reactor, said method comprising removing the one or more retro-aldol catalysts from the reactor whilst also in the presence of one or more agents suitable to suppress tungsten precipitation.

2. The method according to claim 1, wherein the one or more agents suitable to suppress tungsten precipitation comprise at least one of organic oxygenates or buffer systems comprising one or more organic acids, their corresponding conjugated bases with alkali-metal as their counterions, and mixtures thereof.

3. The method according to claim 2, wherein the one or more agents suitable to suppress tungsten precipitation comprise at least one of organic oxygenate solvents, the stream comprising one or more saccharides, glycols, sugar alcohols, carboxylic acids, other products formed during the process, and mixtures thereof.

4. The method according to claim 1, wherein the one or more retro-aldol catalysts are removed from the reactor prior to or along with removal of the one or more agents suitable to suppress tungsten precipitation.

5. The method according claim 1, wherein the reactor temperature is lowered to less than 160° C. prior to the removing the one or more retro-aldol catalysts.

6. The method according to claim 1, wherein the stream comprising one or more saccharides continues to be fed to the reactor after the removal of the one or more retro-aldol catalysts.

7. The method according to claim 1, wherein the stream comprising one or more saccharides are selected from the group consisting of monosaccharides, disaccharides, oligosaccharides, polysaccharides, or a mix of these.

8. The method according to claim 1, wherein the one or more retro-aldol catalysts comprise at least one of silver tungstate, sodium metatungstate, sodium tungstate, ammonium metatungstate, sodium polytungstate, tungstic acid, alkali and alkaline earth metal tungstates, alkali and alkaline earth phosphotungstates, phosphotungstic acid, mixed tungstates and molybdates and silicotungstic acid.

9. A shutdown process for the preparation of monoethylene glycol from a stream comprising one or more saccharides in the presence of hydrogen and a catalyst system comprising one or more tungsten based retro-aldol catalysts in a reactor comprising one or more catalytic species suitable for hydrogenation, said process comprising:
a. reducing the reactor temperature to less than 160° C.;
b. removing the one or more tungsten based retro-aldol catalysts from the reactor; and
c. removing the one or more agents suitable to suppress tungsten precipitation from the reactor.

10. The process according to claim 9, wherein the removing of the one or more retro-aldol catalysts occurs after or concurrently with the reducing the reactor temperature.

11. The process according to claim 9, wherein the removing of the one or more retro-aldol catalysts occurs prior to or concurrently with removing the one or more agents suitable to suppress tungsten precipitation from the reactor.

12. The process according to claim 9, further comprising removing the one or more saccharides from the reactor after all the one or more tungsten based retro-aldol catalysts have been removed from the reactor.

13. The process according to claim 12, further comprising introducing water to the reactor to remove all of the organics from the reactor.

14. The process according to claim 12, wherein the introducing the water occurs concurrently with the removing the one or more saccharides from the reactor.

15. The process according to claim 9, wherein the one or more agents suitable to suppress tungsten precipitation comprise at least one of organic oxygenate solvents, glycols, sugar alcohols, or buffer systems comprising one or more organic acids, their corresponding conjugated bases with alkali-metal as their counterions, and mixtures thereof.
